(19) United States
(12) Patent Application Publication
(10) Pub. No.: US 2010/0191184 A1
(43) Pub. Date: Jul. 29, 2010

(54) SYRINGE WITH ADJUSTABLE NEEDLE

(76) Inventor: Jong-Soo Choi, Seoul (KR)

Correspondence Address: HARNESS, DICKEY & PIERCE, P.L.C., P.O. BOX 8910, RESTON, VA, 20195, US

(21) Appl. No.: 12/669,349

(22) PCT Filed: Oct. 8, 2007
(86) PCT No.: PCT/KR07/04889

§ 371 Date: Jan. 15, 2010

(30) Foreign Application Priority Data
Jul. 27, 2007 (KR) 10-2007-0075657

(51) Int. Cl. A61M 5/46

(52) U.S. Cl. 604/117

(57) ABSTRACT

A syringe capable of adjusting the length of a needle is disclosed. The syringe includes a barrel filled with an injection therein and having a mounting portion protruding from a front portion of the barrel, a plunger slidably engaged with the barrel for pressing the injection, a needle hub having an inner diameter corresponding to an outer diameter of the mounting portion, the needle hub being forcibly fitted in the mounting portion and provided with a threaded portion at an outer periphery of the needle hub, and an adjusting member threadedly engaged with the outer periphery of the needle hub for adjusting the length of the exposed needle. The adjusting member can be produced in mass by simply changing the design of an injection mold, which can reduce a production cost in comparison to a conventional way in which needles in various specifications should be produced. When the adjusting member is mounted in a kind of syringe, it is convenient to repeatedly inject the injection in a desired depth.

### TECHNICAL FIELD

The present invention relates to a syringe, and more particularly, to a syringe with an adjustable needle which can freely adjust a length of the needle depending upon a portion to be injected of a patient.

### BACKGROUND ART

Injection is generally classified into intramuscular injection, intravenous injection, hypodermic injection, and the like, according to injected portions of humans or animals in hospitals. Injection procedure performed by doctors or nurses is regulated by laws related to health measure and hygiene which should utilize a needle assembly consisting of a needle and a needle hub standardized to purposes.

Therefore, hospitals are generally equipped with large needles for intramuscular injection, middle needles for intravenous injection, and small needles for hypodermic injection, respectively. Needle manufacturers make and supply sets of packaged needles to standards In this instance, general syringes that are commonly used and commercially available are classified into two types which will be described hereinafter.

FIG. 1 shows a general syringe that is commonly used and commercially available.

Referring to FIG. 1, a barrel 11 is formed in a cylinder shape, in which a plunger 12 is moved back and forth therein. A threaded portion 11a is formed on a front inner periphery of the barrel 11 to assemble a needle assembly 30. A plunger ring 12a is coupled to the front portion of the plunger to expulse an injection, and a knob 12b is integrally formed on the rear portion of the plunger. A flange 11b is provided at a rear portion of the barrel 11 in order to apply counteraction force to the barrel 11 when a user pushes or pulls the knob.

The needle assembly 30 provided in various sizes (e.g., 30A, 30B, and 300) includes a needle 31 and a needle hub 32 fixing a rear end of the needle 31 and assembled to or disassembled from the threaded portion of the syringe 10. The needle hub 32 is provided with a plurality of anti-slid bosses 33 formed on the outer periphery thereof in order to prevent slippage when the user assembles the needle hub to the threaded portion 11a or disassembles it therefrom, while holding it by his or her hands. An assembling boss 34 is formed on the outer periphery of the needle hub 32 which is mounted on the threaded portion 11a, so as to prevent the injection from being leaked due to assembling resiliency.

In addition, a sealing 40 is interposed between the needle hub 32 and the barrel 11 so as to prevent fluid tightness there between when the needle hub 32 is engaged to the barrel 11, which is hereinafter referred to as a threaded engagement type syringe.

FIG. 2 shows another syringe 20 including a needle assembly different from the threaded engagement type syringe 10, in which the needle assembly is indicated by the same reference numeral as that in FIG. 1 since the construction is substantially identical to each other.

A mounting portion 21a protrudes from a front end of the barrel 21 in a predetermined length, and has a diameter smaller than that of the barrel 21.

More specifically, the syringe 20 includes the mounting portion 21a having the diameter smaller than that of the barrel 21, so that an outer periphery of the mounting portion 21a is forcibly fitted in an inner periphery of the needle hub 32 in the process of assembling the needle assembly 30.

The syringe further includes a plunger 22 moved back and forth in the barrel 21, a plunger ring 22a coupled to the front portion of the plunger 22, a knob 22b formed on an end of the plunger 22, and a flange 22b provided at a rear portion of the barrel 21 in order to apply operating force to the knob 22b, which is hereinafter referred to as a forced fitting type syringe.

However, since prices of the needles 31 applied to the general syringes are different depending upon a size thereof, some of hospitals are equipped with one kind of inexpensive needles 31 to inoculate patients. For example, there is a case of utilizing a large needle when injection is carried out for a skin layer in very shallow depth in dermatology or dentistry. In this instance, doctors or nurses should carefully inject so that the needle is not inserted into the skin in excessive depth. Therefore, it may increase the fatigue of operators

Also, in case in which a patient recognizes the protruding needle in the process of performing an operation, the patient may feel uneasy about the needle.

### DISCLOSURE

### Technical Problem

Therefore, an object of the present invention is to solve the problems involved in the prior art, and to provide syringe with an adjustable needle which can adjust a protruding length of the needle by covering the needle with an adjusting member under circumstances having no proper needles.

### Technical Solution

In order to achieve these and other objects, the present invention provides a syringe capable of adjusting a length of a needle is disclosed. The syringe includes a barrel filled with an injection therein and having a mounting portion protruding from a front portion of the barrel, a plunger slidably engaged with the barrel for pressing the injection, a needle hub having an inner diameter corresponding to an outer diameter of the mounting portion, the needle hub being forcibly fitted in the mounting portion and provided with a threaded portion at an outer periphery of the needle hub, and an adjusting member threadedly engaged with the outer periphery of the needle hub for adjusting a protruding length of the needle.

### ADVANTAGEOUS EFFECTS

The adjusting member can be produced in mass by simply changing a design of an injection mold, which can reduce a production cost in comparison with a conventional way which should produce needles in various specifications. When the adjusting member is mounted in a syringe of one kind, it is convenient to repeatedly inject the injection in a desired depth.

### DESCRIPTION OF DRAWINGS

The above objects, other features and advantages of the present invention will become more apparent by describing the preferred embodiment thereof with reference to the accompanying drawings, in which:

FIG. 1 is a cross-sectional view illustrating a conventional syringe.

FIG. 2 is a cross-sectional view illustrating another conventional syringe.

FIG. 3 is an exploded perspective view illustrating a syringe according to a first embodiment of the present invention.

FIG. 4 is a cross-sectional view of the syringe in FIG. 3.

FIG. 5 is an exploded perspective view illustrating a syringe according to a second embodiment of the present invention.

FIG. 6 is a cross-sectional view of the syringe in FIG. 5.

FIG. 7 is an exploded perspective view illustrating a syringe according to a third embodiment of the present invention.

FIG. 8 is a cross-sectional view of the syringe in FIG. 7.

FIG. 9 is an exploded perspective view illustrating a syringe according to a fourth embodiment of the present invention.

FIG. 10 is a cross-sectional view of the syringe in FIG. 9.

FIG. 11 is an exploded perspective view illustrating a syringe according to a fifth embodiment of the present invention.

FIG. 12 is a cross-sectional view of the syringe in FIG. 11.

FIG. 13 is an exploded perspective view illustrating a syringe according to a sixth embodiment of the present invention.

FIG. 14 is a cross-sectional view of the syringe in FIG. 13.

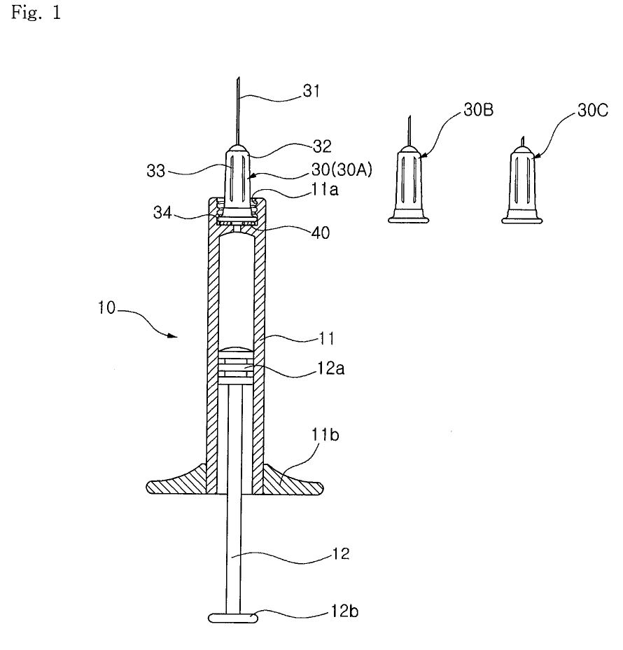

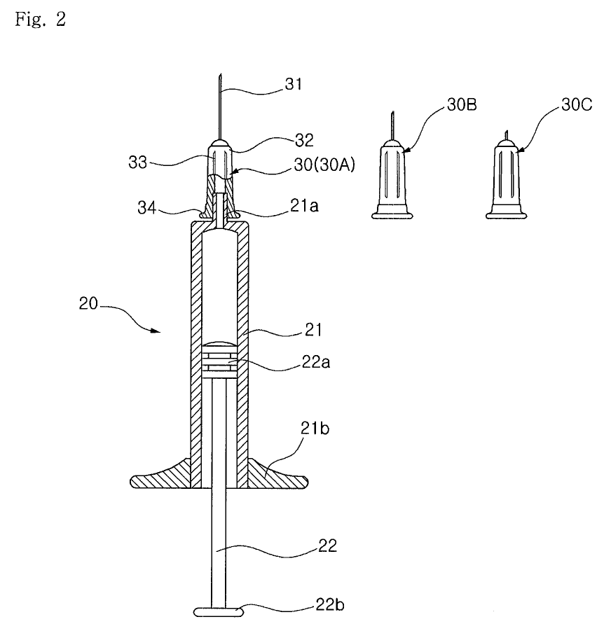

### BEST MODE

Reference will now be made in detail to preferred embodiments of the present invention, examples of which are illustrated in the accompanying drawings.

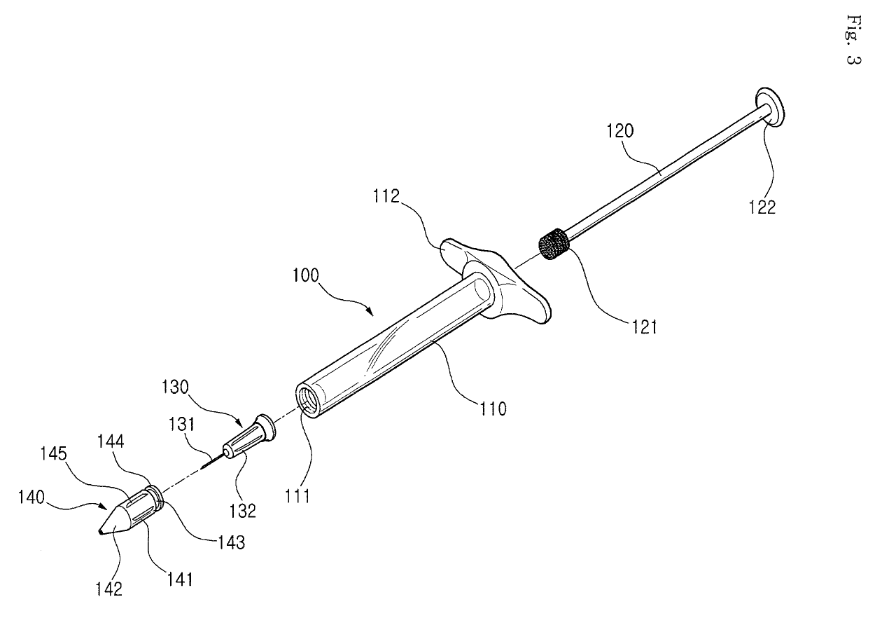

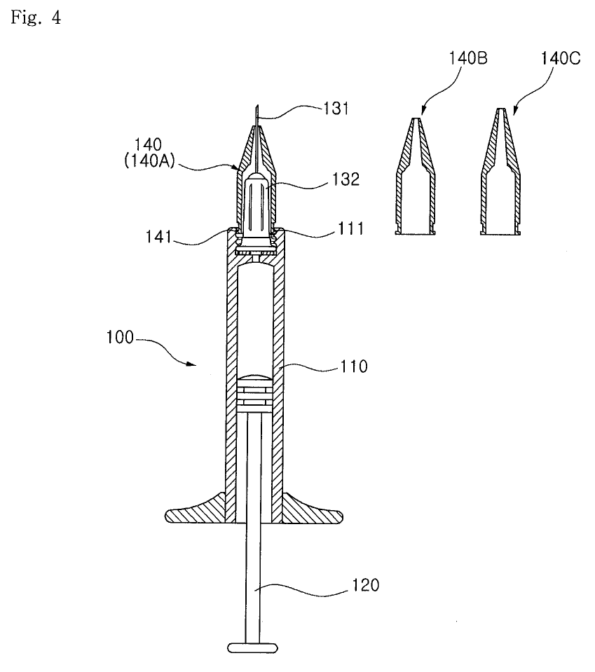

FIGS. 3 and 4 show the construction of a syringe according to a first embodiment of the present invention.

Referring to the drawings, a syringe 100 of this embodiment is a threaded engagement type syringe, and includes a barrel 111 of a cylinder shape and a plunger 120 inserted into the barrel 110. A plunger ring 121 of rubber is coupled to the front portion of the plunger 120, and a knob 122 is formed on the rear portion of the plunger 120, and has a diameter larger than that of the plunger in order to press the plunger 120.

A threaded portion 111 is formed on the front inner periphery of the barrel 110 to assemble a needle assembly 30 consisting of a needle 131 and a needle hub 132. Another needle hub 132 may be assembled to the threaded portion 111.

The syringe further includes an adjusting member 140 for enclosing a portion of the needle assembly 130. In this embodiment, adjusting members 140A, 140B and 140C may be applied to adjust a length of the needle 131, while a large needle 131 is mounted in the barrel 110. The adjusting member 140 includes a hollow body 141, a front taper portion 142 integrally formed on a front portion of the body 141 and having a diameter gradually decreased towards an end of the needle 131, a fitting portion 143 formed on a rear portion of the body 141 and having an outer diameter smaller than that of the body, and a locker 144 protruding from a lower end of the fitting portion and threadedly engaged with the threaded portion 111 of the syringe. In this instance, an anti-skid boss 145 is formed on an outer periphery of the body 141 in a spline pattern.

There is no modification in the syringe 100 and the needle assembly 130 to apply the adjusting members 140A, 140B, and 140C. More specifically, the lower ends of the adjusting members 140A, 140B, and 140C have an outer meter in such a way that the adjusting members are threadedly engaged to the front end of the barrel 110.

The adjusting member 140 can be applied to adjust the wanted length to of the needle in the state a large needle is mounted on the barrel 110. In order to select the wanted length of the needle, therefore, any one of the adjusting members 140 having various sizes (e.g., 140A, 140B, and 140C) is selected to be mounted on the treaded portion 111 of the syringe.

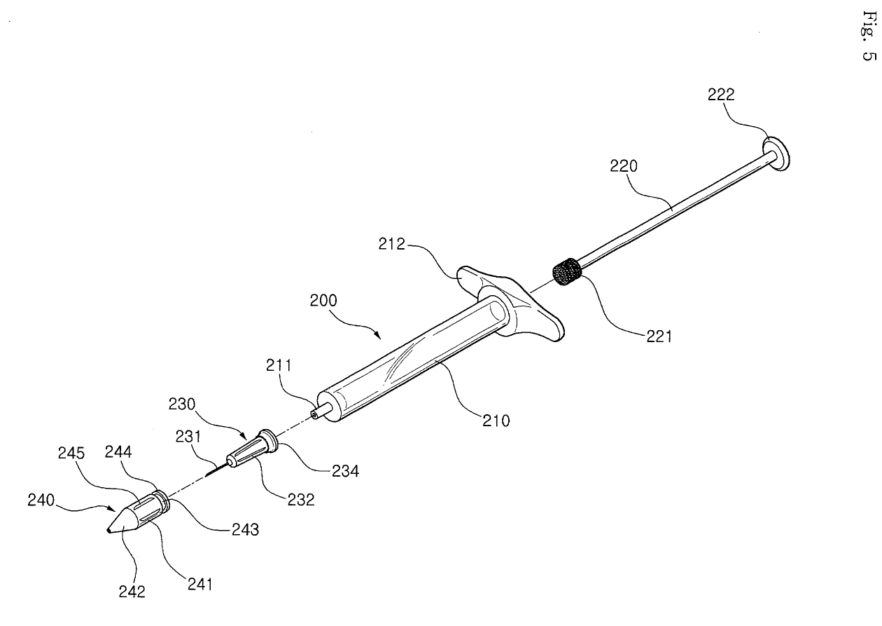

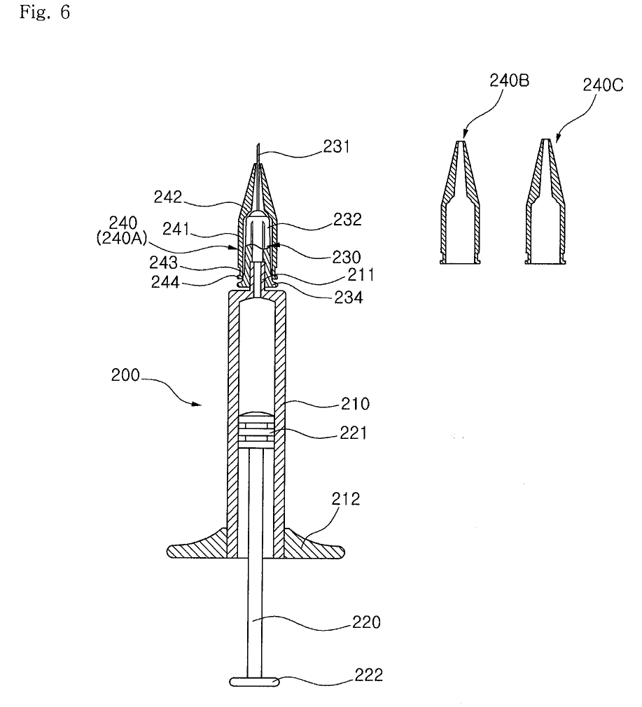

FIGS. 5 and 6 show the construction of a syringe according to a second embodiment of the present invention.

Referring to the drawings, a syringe 200 of this embodiment is a forced fitting type syringe, and includes a barrel 211 of a cylinder shape, a plunger 220 inserted into the barrel 210, and a needle 231 protruding from a front end of a needle hub 230.

In this instance, a mounting portion 211 protrudes from the front end of the barrel 210 to have a predetermined length, and has an outer diameter smaller than that of the barrel 210.

In addition, the syringe further includes an adjusting member 240 for adjusting a protruding length of the needle 231, which is identical to the construction shown in the first embodiment.

More specifically, there is no modification in the syringes 100 and 200 and the needle assemblies 130 and 230 to apply the adjusting members 140 and 240, according to the first and second embodiments.

In order to apply the adjusting member to both the syringes 100 and 200 of two kinds, of which the shapes are different to each other, the inner diameter and the outer diameter are set in the cylindrical adjusting members 140 and 240 according to the first and second embodiments. To this end, the outer diameter of each lower end of adjusting members 140 and 240 is set in such a way that it can be threadedly engaged with the inner diameter portion of the barrel 110 of the threaded engagement type syringe. Also, the inner diameter of each lower end of each adjusting members 140 and 240 is set in such a way that it can be forcibly fitted into the assembling boss 34 of the needle hub 32 which is mounted in the barrel 110 of the forcible fitting type syringe.

The reason why the adjusting members 140 and 240 are mounted on the front end of the syringes 100 and 200 is that the length of the needle protruding from the front end of the syringe is adjusted by using a component which is constant in cost according to its size, instead of replacing it with the needle assembly 130 or 230 of different size, of which the cost is different from each other according to its size.

More specifically, in state the needle hub 130 or 230 is previously assembled to the front end of the barrel 110 or 210, the adjusting member can be assembled or disassembled to forwardly expose only a portion of the needle 131 or 231 from the front portion of the adjusting member.

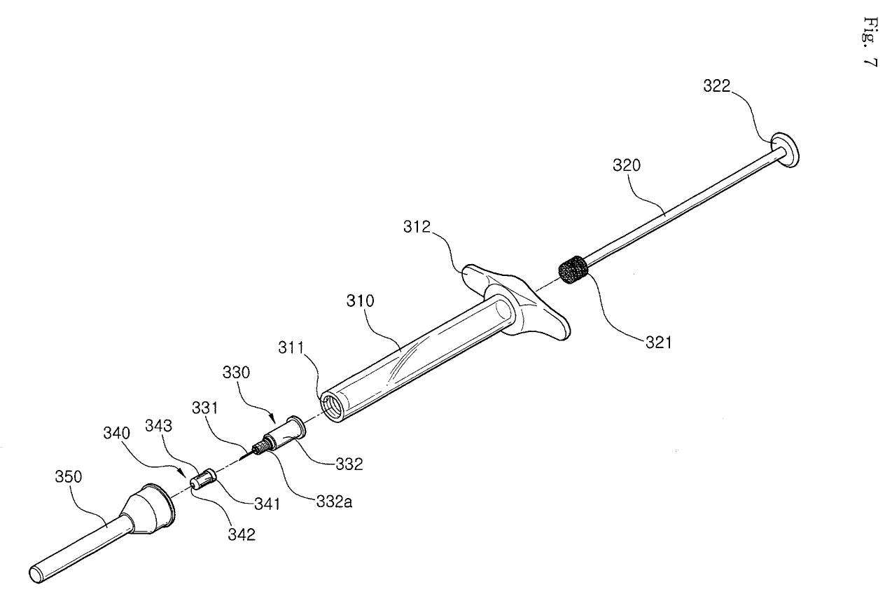

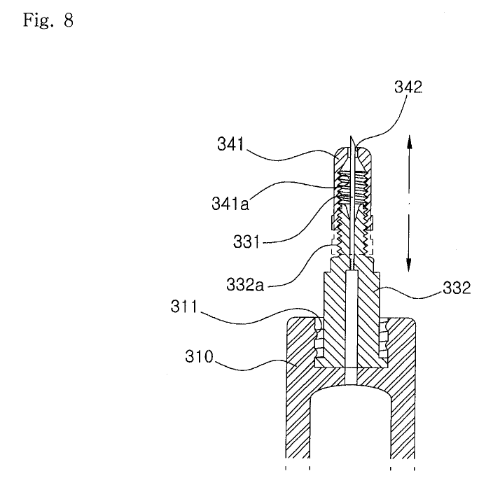

FIGS. 7 and 8 show the construction of a syringe according to third second embodiment of the present invention.

Referring to the drawings, a syringe 300 of this embodiment is a threaded engagement type syringe, in which since the construction of the syringe is substantially identical to that in the first embodiment, it is not described herein.

Meanwhile, the syringe further includes a protective cap 350 easily fitted in the front portion of a barrel 310 for preventing a needle 331 from being exposed outwardly after the syringe 300 is manufactured. The protective cap 350 prevents damage of the needle 331 and an accident from happening due to the needle 331.

In this embodiment, an adjusting member 340 is provided on the outside of a needle hub 330 when it is necessary to adjust the protruding length of the needle 331 in the syringe 300 according to operation circumstance. The adjusting member 340 includes a cylindrical hollow body 341, and a through-hole 342 is formed in a center of the upper surface of the body 341.

In this embodiment, the adjusting member 340 is not mounted on the barrel 310, but is engaged to the outside of the needle hub 332, contrary to the first and second embodiments.

More specifically, a treaded portion 332a is formed on the outer periphery of the needle hub 332, and a threaded portion 341a is formed on the rear inner periphery of the adjusting member 340.

In this instance, the adjusting member 340 has an outer diameter corresponding to the outer diameter of the needle hub 332, and the needle hub 332 has an outer diameter to be received in the protective cap 350. Therefore, it is not necessary to modify the shape of a conventional syringe or needle 331 in order to apply the adjusting member 340.

Meanwhile, only one adjusting member is necessary in this embodiment, contrary to the first and second embodiments requiring several kinds of the adjusting members.

The reason why the adjusting member 340 is mounted on the outside of the needle hub 332 is that the length of the needle 331 protruding from the front end of the syringe is adjusted by using a component which is constant in cost according to its size, instead of replacing the needle hub 332 having the needle 331, of which the cost is different from each other according to its size. That is, the length of the needle 331 protruding from the front end of the syringe is adjusted by using the adjusting member 340. More specifically, in state the needle hub 332 is previously assembled to the front end of the barrel 310, the adjusting member can be assembled or disassembled to forwardly expose only a portion of the needle 331 from the front portion of the adjusting member 340.

Meanwhile, an anti-skid boss 343 is formed on an outer periphery of the adjusting member 340 in a spline pattern.

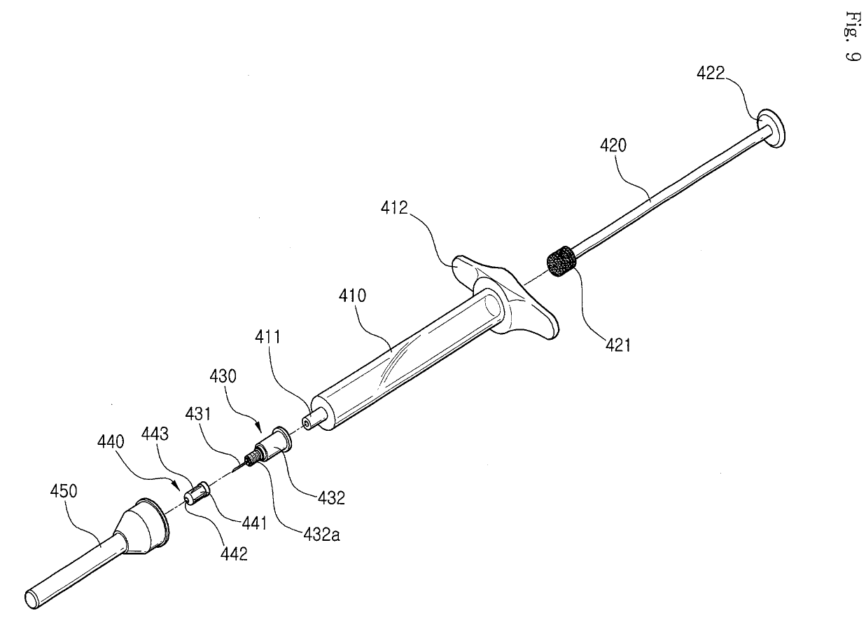

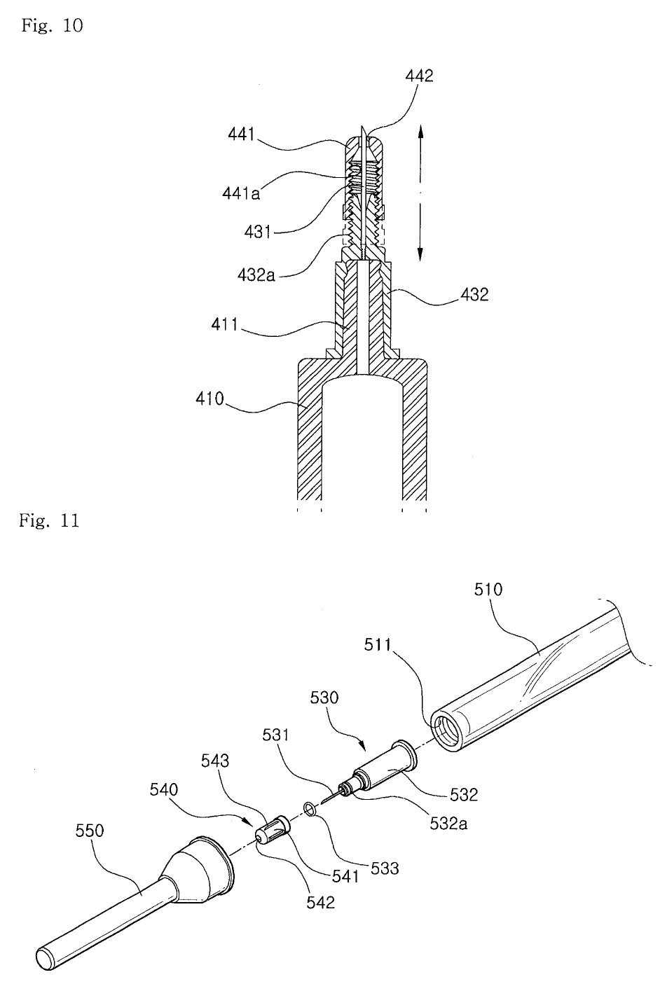

FIGS. 9 and 10 show the construction of a syringe according to a fourth embodiment of the present invention.

Referring to the drawings, a syringe 400 of this embodiment is a forced fitting type syringe, in which since the construction of the syringe is substantially identical to that in the fourth embodiment, it is not described herein.

The syringe 400 further includes a needle hub 432 having an inner diameter corresponding to a mounting portion 411 which protrudes from a front end of a barrel 410. An adjusting member 440 having a threaded portion 432a is engaged to the threaded portion 432a of the needle hub 432 to adjust the protruding length of the needle 431.

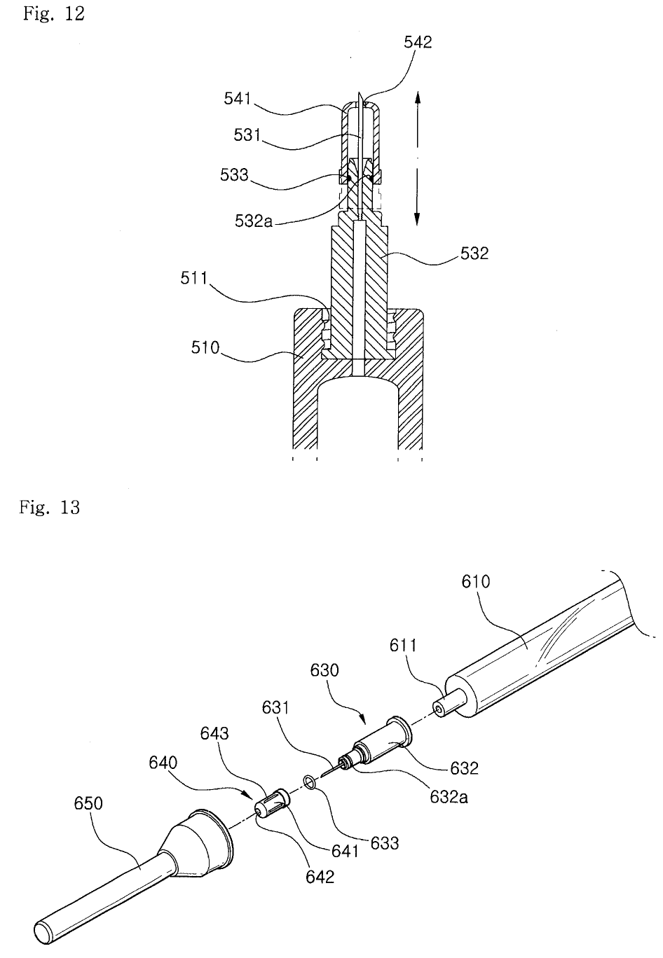

The adjusting member 440 having a threaded portion 441a formed on the inner periphery thereof is engaged to the threaded portion 432a of the needle hub 432 to adjust the protruding length of the needle 431. FIGS. 11 and 12 show the construction of a syringe according to a fifth embodiment of the present invention.

In this embodiment, since the construction of the syringe is similar to that in the third embodiment, it is not described herein.

However, the corresponding threaded portions are formed on the outer periphery of the needle hub and the inner periphery of the adjusting member in this embodiment, but a seating groove 532a is formed on the outer periphery of a needle hub 532 in this embodiment, in which an O-ring 533 is fitted into the seating groove 532a. Preferably, the O-ring 533 is made of soft material, such as rubber, in order to prevent scratch or damage form happening in the adjusting member 540, since the O-ring 533 comes in contact with the inner periphery of the adjusting member 540.

With the above construction, an operator presses the adjusting member 540 forward or rearward to protrude the needle 531 by necessary length, with the inner periphery of the adjusting member 540 contacting the O-ring 533, so that the protruding length of a needle 531 is adjusted. Therefore, it is required for a process of forming the seating groove 532 on the needle hub 532, without performing tap machining on the needle hug 532 and the adjusting member 540 to form the threaded portions, which can reduce the manufacturing process and cost relative to the first to fourth embodiments.

The syringe of this embodiment includes the O-ring 533, but it is not limited thereto. Alternatively, a rubber contact boss (not shown) may be formed on the outer periphery of the needle hub 532.

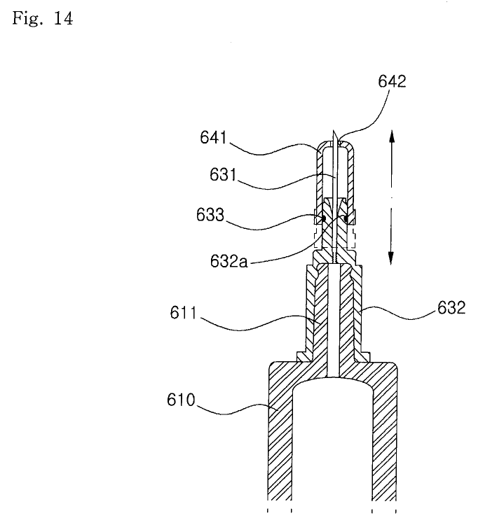

FIGS. 13 and 14 show the construction of a syringe according to a sixth embodiment of the present invention.

In this embodiment, the construction of a syringe 600 is similar to that in the fourth embodiment, and the construction of an adjusting member 640 is identical to that in the fifth embodiment.

More specifically, an O-ring 633 made of rubber is fitted into a seating groove 632a formed on the outer periphery of a needle hub 632, with a needle hub 632 being forcibly fitted into a mounting portion 611 of a barrel 610. An adjusting member 640 is engaged to the outside of the needle hub 632 to come into contact with the O-ring 633.

As described above, in case of using a general syringe, doctors or nurses should mount the syringe on the needle hub having the needle of a proper length according to a portion to be injected or injection purpose. However, the present invention can perform diverse injection by using the adjusting member, with only the needle hub having any needle being mounted on the syringe. More specifically, in case of using a middle or large needle, the length of the needle protruding from the front portion of the syringe can be adjusted by using the adjusting member.

For example, a doctor or nurse firstly mounts a selected needle onto the threaded portion or mounting portion provided on the barrel, and sticks the needle into an ampule. Then, the injection is sucked in the barrel by pulling the knob exposed at the rear portion of the barrel.

The adjusting member is threadedly engaged to the threaded portion of the threaded engagement type syringe or mounted on the mounting portion of the forcible fitting type syringe, in order to adjust the protruding length of the needle.

While the present invention has been described and illustrated herein with reference to the preferred embodiments thereof, it will be apparent to those skilled in the art that various modifications and variations can be made therein without departing from the spirit and scope of the invention. Thus, it is intended that the present invention covers the modifications and variations of this invention that come within the scope of the appended claims and their equivalents.

### INDUSTRIAL APPLICABILITY

The adjusting member can be produced in mass by simply changing a design of an injection mold, which can reduce a production cost in comparison with a conventional way which should produce needles in various specifications. When the adjusting member is mounted in a syringe of one kind, it is convenient to repeatedly inject the injection in a desired depth.

1. A syringe capable of adjusting a length of a needle, comprising: a barrel filled with an injection therein and having a threaded portion formed on a front inner periphery of the barrel;
a plunger slidably engaged with the barrel for expelling the injection out of the barrel;
a needle hub having an assembling boss formed on an outer periphery thereof which is threadedly engaged with the threaded portion of the barrel, a needle being fixed to a center portion of the needle hub; and
a cylindrical adjusting member enclosing an outer portion of the needle hub and having a locker which is fastened to the threaded portion of the barrel.

2. A syringe capable of adjusting a length of a needle, comprising:
a barrel filled with an injection therein and having a mounting portion protruding from a front portion of the barrel;
a plunger slidably engaged with the barrel for expelling the injection out of the barrel;
a needle hub forcibly fitted into the mounting portion of the barrel, a needle being fixed to a center portion of the needle hub; and
a cylindrical adjusting member forcibly fitted around the needle hub.

3. The syringe as claimed in claim 1, wherein the adjusting member has a diameter in such a way that the adjusting member is forcibly fitted between a body of the needle hub and the assembling boss.

4. The syringe as claimed in claim 1, wherein the adjusting member includes a taper portion with a diameter gradually decreased towards an end of the needle.

5. The syringe as claimed in claim 1, wherein an anti-skid boss is formed on an outer periphery of the adjusting member in a spline pattern.

6. A syringe capable of adjusting a length of a needle, comprising:
a barrel filled with an injection therein and having a threaded portion fouled on a front inner periphery of the barrel;
a plunger slidably engaged with the barrel for expelling the injection out of the barrel;
a needle hub having an assembling boss formed on an outer periphery thereof which is threadedly engaged with the threaded portion of the barrel, and a threaded portion formed on the outer periphery, a needle being fixed to a center portion of the needle hub; and
an adjusting member threadedly engaged with the outer periphery of the needle hub for adjusting a protruding length of the needle.

7. A syringe capable of adjusting a length of a needle, comprising:
a barrel filled with an injection therein and having a mounting portion protruding from a front portion of the barrel;
a plunger slidably engaged with the barrel for expelling the injection out of the barrel;
a needle hub having an inner diameter corresponding to an outer diameter of the mounting portion to forcibly fit the needle hub into the mounting portion of the barrel, and a threaded portion formed on the outer periphery, a needle being fixed to a center portion of the needle hub; and
an adjusting member threadedly engaged with the outer periphery of the needle hub for adjusting a protruding length of the needle.

8. The syringe as claimed in claim 6, wherein an anti-skid boss is formed on an outer periphery of the adjusting member in a spline pattern

9. A syringe capable of adjusting a length of a needle, comprising:
a barrel filled with an injection therein and having a threaded portion formed on a front inner periphery of the barrel;
a plunger slidably engaged with the barrel for expelling the injection out of the barrel;
a needle hub having an assembling boss formed on an outer periphery thereof which is threadedly engaged with the threaded portion of the barrel, and a seating groove formed on the outer periphery, a needle being fixed to a center portion of the needle hub, and an O-ring being fitted into the seating groove; and
an adjusting member forcibly engaged with the outer periphery of the needle hub which comes in contact with the O-ring, for adjusting a protruding length of the needle.

10. A syringe capable of adjusting a length of a needle, comprising:
a barrel filled with an injection therein and having a mounting portion protruding from a front portion of the barrel;
a plunger slidably engaged with the barrel for expelling the injection out of the barrel;
a needle hub having an inner diameter corresponding to an outer diameter of the mounting portion to forcibly fit the needle hub into the mounting portion of the barrel, and a seating groove formed on the outer periphery, a needle being fixed to a center portion of the needle hub, and an O-ring being fitted into the seating groove; and
an adjusting member forcibly engaged with the outer periphery of the needle hub for adjusting a protruding length of the needle.

11. The syringe as claimed in claim 9, wherein an anti-skid boss is formed on an outer periphery of the adjusting member in a spline pattern.
